(19) United States
(12) Patent Application Publication
(10) Pub. No.: US 2026/0107959 A1
(43) Pub. Date: Apr. 23, 2026

(54) HYBRID CULTURED MEAT PRODUCTS AND RELATED METHODS

(71) Applicant: The Trustees of Boston College, Chestnut Hill, MA (US)

(72) Inventors: Glenn R. GAUDETTE, Holden, MA (US); Richard THYDEN, Sutton, MA (US); Antonio Carlos FREITAS DOS SANTOS, Waltham, MA (US); David REDDIG, Brownstown, PA (US); Joseph Thomas LIZEWSKI, Easton, MD (US)

(21) Appl. No.: 19/364,844

(22) Filed: Oct. 21, 2025

Related U.S. Application Data
(60) Provisional application No. 63/709,848, filed on Oct. 21, 2024.

(51) Int. Cl. A23L 13/40; C12N 5/00; C12N 5/04; C12N 9/10

(52) U.S. Cl. CPC A23L 13/426; C12N 5/0025; C12N 5/04; C12N 9/1044; C12N 2502/1335; C12Y 203/02013

(57) ABSTRACT

The method involves producing a hybrid cultured meat product by decellularizing plant scaffolds through sequential exposure to decellularizing agents and rinsing to achieve a low residual DNA level. The decellularized plant scaffolds are seeded with mammalian or poultry cells or cells derived therefrom. The seeded decellularized plant scaffolds and then mixed with a protein source and a binding agent to form a mixture, which is shaped into the desired form. The process may include specific treatments with sodium dodecyl sulfate and bleach, and the use of a protein source and transglutaminase as components. The resulting product offers unique textures and nutritional values from the combination of plant scaffolds and protein sources.

### CROSS REFERENCE TO RELATED APPLICATIONS

This application claims the benefit of priority to U.S. Provisional Application Ser. No. 63/709,848, filed Oct. 21, 2024, the disclosures of which is incorporated by reference as if fully set forth herein in its entirety.

### TECHNICAL FIELD

The present disclosure relates to a hybrid cultured meat product, and more particularly, to a composition and method for creating a cultured meat product using decellularized plant scaffolds and proteins.

### BACKGROUND

The food industry is constantly evolving to meet the demands of a growing population and the need for sustainable food sources. Traditional meat production has been scrutinized for its environmental impact, including high greenhouse gas emissions, land use, and water consumption. As a result, there has been a significant push towards developing alternative protein sources that are more sustainable and environmentally friendly. Cultured meat, also known as lab-grown or cell-based meat, has emerged as a promising solution to these challenges. It involves cultivating animal cells in a controlled environment to produce meat without the need for raising and slaughtering animals. This approach not only reduces the environmental footprint but also addresses ethical concerns related to animal welfare.

In addition to environmental and ethical considerations, there is a growing interest in enhancing the nutritional profile and textural qualities of alternative protein products. Plant-based proteins have gained popularity due to their health benefits and lower environmental impact compared to animal-based proteins. However, achieving the desired texture and flavor that mimics traditional meat remains a challenge. The integration of plant-based components with cultured meat technologies offers a potential pathway to create hybrid products that combine the best attributes of both worlds. By leveraging the structural properties of plant materials and the nutritional benefits of cultured cells, researchers aim to develop innovative food products that cater to the evolving preferences of consumers seeking sustainable and nutritious alternatives to conventional meat. Although the incorporation of decellularized plants for cultured meat applications have been documented in the literature,¹ there is yet to be an expressed or demonstrated ability for binding decellularized plants together via mammalian cells to create novel edible cultured meat products.

Accordingly, there is a need for hybrid cultured meat products with enhanced nutritional profile and textural qualities that mimics traditional meats.

### SUMMARY

For purposes of summary, certain aspects, advantages, and novel features are described herein. It is to be understood that not necessarily all such advantages may be achieved in accordance with any one particular embodiment. Thus, the apparatuses, systems, or methods claimed may be embodied or carried out in a manner that achieves or optimizes one advantage or group of advantages as taught herein without necessarily achieving other advantages as may be taught or suggested herein.

As mentioned above, although the incorporation of decellularized plants for cultured meat applications have been documented in the literature, there is yet to be an expressed or demonstrated ability for binding decellularized plants together via mammalian cells to create novel edible cultured meat products. The present disclosure provides a hybrid style food that contains both the nutritional characteristics of both the cells and the plants that are bound together. By incorporating scaffolds bound together via cells into the final product, the methods of the present disclosure eliminate the need for a cell harvest step. Moreover, every plant that can be decellularized, when bound to one another via cells, can provide different textural and different nutritional values to the final product. Decellularized plant scaffolds from one organism may be bound to that of another plant, to create more unique textures and more unique marketable products.

The present disclosure relates to hybrid cultured meat products and to methods and systems for making the same. In one aspect, a method of producing a hybrid cultured meat product is provided. The method includes (a) decellularizing plant scaffolds by sequential exposure to decellularizing agents and rinsing to achieve a residual DNA level below a threshold; (b) mixing the decellularized plant scaffolds with a protein source and a binding agent to create a mixture; and (c) forming the mixture into a desired shape to create the hybrid cultured meat product.

Various plants, fruits, and vegetables can be used to make decellularized scaffolds. In certain embodiments, the plant scaffolds comprise plants, fruits, and vegetables selected from broccoli florets, spinach leaves, corn stover, jackfruit rinds, parsley leaves and stem, calathea, vanilla, anthurium, orchid, bamboo, solenostemon (coleus), Artemisia annua (aka sweet wormwood) leaves, peanut hairy roots, seaweed, rockweed, apple peels, banana peels, orange peels, and/or blades of grass. In certain embodiments, the plant scaffolds comprise broccoli, cauliflower, mushrooms, spinach, celery, and apple. In certain embodiments, the plant scaffolds comprise broccoli florets.

The decellularizing agents can comprise sodium dodecyl sulfate (SDS) and bleach. By way of example, the decellularizing can include two SDS (5% w/v) treatments over two days and a bleach (5% w/v) treatment over twenty-four hours. In some instances, the residual DNA level is at or below 0.05 μg/mL, 0.02 μg/mL, 0.01 μg/mL, 0.005 μg/mL. In some instances, the residual DNA level is at or below 0.01 μg/mL, or at or below 2% of fresh plant scaffolds.

In some embodiments, the decellularized plant scaffolds are seeded with mammalian or poultry cells. Numerous types of cells are suitable for this purpose including, but not limited to, (i) primary skeletal muscle satellite cells or myoblasts from bovine, porcine, ovine, and/or poultry; (ii) adipogenic precursors: preadipocytes or adipose-derived stromal/stem cells (ADSCs) for lipid deposition and flavor; (iii) mesenchymal stromal/stem cells (MSCs): multipotent progenitors that can be directed to myogenic, adipogenic, or fibrogenic lineages; (iv) fibroblasts and connective-tissue progenitors: to tune texture via extracellular matrix deposition; (v) endothelial or endothelial-like cells: optional for co-culture support or perfusability studies; (vi) induced pluripotent stem cell (iPSC)-derived myogenic or adipogenic progenitors: scalable sources for manufacturing; and (vii) co-cultures (e.g., myogenic+adipogenic±fibroblasts/endothelial) to approximate whole-muscle composition. In some embodiments, the decellularized plant scaffolds is seeded with QM7 cells, a quail myoblast line that was originally established from the pectoral muscle of a Japanese quail embryo.

In some embodiments, the protein source is derived from one or more of peas, soybeans, wheat, chickpeas, lentils, and mycoprotein, or a combination thereof. In some embodiments, the protein source comprises pea protein. This protein source is in addition to the proteins derived from the above-described mammalian or poultry cells seeding the decellularized plant scaffolds.

In some embodiments, the binding agent comprises is selected from one or more of transglutaminase, alginate+Ca²⁺ (0.5-2.0%), Gellan gum (0.1-0.6%), K-Carrageenan (works well with pea/soy matrices at ˜0.5-2%), methylcellulose (0.5-3.0%), low-methoxyl pectin+Ca²⁺ (0.5-2.0% pectin; 5-30 mM Ca²⁺), konjac glucomannan+xanthan (0.2-1.0% KGM+0.05-0.2% xanthan), laccase+pectin (50-500 U/kg laccase; 0.2-1% pectin), tyrosinase (polyphenol oxidase; 20-200 U/kg), wheat gluten (3-8%), sodium caseinate (0.5-2.0%), egg albumen (1-3%), and plant fiber concentrates (e.g., citrus/apple/pea; 0.5-3.0%). In some embodiments, the binding agent comprises transglutaminase. Transglutaminase can be added to the mixture before and after drying. In certain examples, the mixture comprises about 1.00 g (wet) decellularized plant scaffolds, about 0.25 g protein source, and about 0.10 g binding agent added before drying, and about 0.10 g binding agent can be added after drying.

The method may further include processing the mixture on an incubator-shaker at about 100 rpm and about 37° C. for about 24 hours. Drying the mixture can include freeze-drying for 24-72 hours and optionally air-drying for 24-120 hours to obtain a moldable mass. In certain variations, the binding agent is kneaded into the moldable mass prior to forming.

In another aspect, a hybrid cultured meat product produced by any of the foregoing methods is provided. The hybrid cultured meat product can exhibit unique textures and nutritional values derived from the combination of the seeded decellularized plant scaffolds and the protein source.

In a further aspect, a system for producing a hybrid cultured meat product is provided. The system includes (a) means for decellularizing plant scaffolds by sequential exposure to decellularizing agents and rinsing to achieve a residual DNA level below a threshold; (b) means for seeding the decellularizing plant scaffolds; (c) means for means for mixing the seeded decellularized plant scaffolds with a protein source and a binding agent to create a mixture; and (d) means for forming the mixture into a desired shape to create the hybrid cultured meat product. In some arrangements, the means for decellularizing plant scaffolds comprise one or more containers for holding the decellularizing agents and the plant scaffolds, and a rinsing apparatus for removing the decellularizing agents from the plant scaffolds.

Features of the foregoing aspects may be combined or practiced separately as appropriate. These and other aspects, embodiments, and advantages will be apparent from the description herein and the appended claims.

Other implementations are also described and recited herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For the purpose of illustration, certain embodiments of the present disclosure are shown in the drawings described below. It should be understood, however, that the invention is not limited to the precise arrangements, dimensions, and instruments shown. In the drawings:

FIG. 1 illustrates a process for creating hybrid cultured meat using plant scaffolds and protein sources.

FIG. 2 illustrates a hybrid cultured meat production system with decellularization, mixing, binding, and forming units.

FIG. 3 is a high-level block diagram illustrating the general components and process for producing edible cultured meat.

FIG. 4 is a bar graph showing the average DNA content (μg/mL) in fresh broccoli florets (left bar) and the decellularized broccoli florets (right bar). Bars indicate standard error of the mean (SEM).

FIG. 5 shows an image of PLL-coated florets loaded with the highest dose of cells (top image) and an image of PLL-coated florets loaded with lowest dose of cells (lower image).

FIG. 6 is a bar chart showing the share of seeded florets at different cell densities, comparing a control group with a group treated with poly-L-lysine (PLL).

FIG. 7 shows a fabricated meatball with associated composition and size.

FIG. 8 is a graph showing force versus displacement curves from compression tests, comparing the mechanical properties of the fabricated hybrid cultured meat product with those of store-bought meatballs.

FIG. 9 shows a fabricated meatball with 24 mm diameter.

FIG. 10 is a composite image showing a color comparison of multiple embodiments of the hybrid cultured meat product with varying amounts of beetroot extract for color, compared to a conventional real meat product.

FIG. 11 presents larger, magnified closeup images permitting a direct visual comparison between a conventional meat product on the left and the fabricated hybrid cultured meat product on the right.

FIG. 12 provides full meatball images of a conventional raw meatball on the left and the fabricated hybrid cultured meatball product on the right.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

As used in this specification and the appended claims, the singular forms “a,” “an” and “the” include plural referents unless the content clearly dictates otherwise. For example, reference to “a cell” includes a combination of two or more cells, and the like.

As used in this specification and the appended claims, the phrase “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The term “statistically significant” or “significantly” refers to statistical significance and generally means a two standard deviation (2SD) or greater difference.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, animal models, (engineered or genetically modified) cells, organoids, constructs, vectors, carriers, adjuvants, compounds, drug delivery system, antibodies and derivatives, vaccines, and reagents, etc., described herein and as such can vary. The terminology used herein is for the purpose of describing particular embodiments only and is not intended to limit the scope of the present invention, which is defined solely by the claims.

Other terms are defined herein within the description of the various aspects of the invention.

### Feeding the World while not Killing it

Feeding a growing global population while reducing environmental impacts is a major challenge. United Nations demographic projections indicate that the world's population will reach approximately 9.7 billion by 2050.² Conventional animal agriculture is a significant contributor to greenhouse gas (GHG) emissions and land use. A comprehensive life-cycle assessment by the Food and Agriculture Organization of the United Nations (FAO) estimates that livestock supply chains emit about 7.1 gigatonnes CO₂-equivalent annually-approximately 14.5% of global anthropogenic GHG emissions.³ Livestock production is also a leading source of methane (CH₄) and nitrous oxide (N₂O). FAO reports that agriculture contributes roughly 40% of anthropogenic methane, with livestock accounting for ˜32% (primarily via enteric fermentation and manure management).⁴ Earlier assessments based on IPCC methods have attributed on the order of ˜40-50% of anthropogenic CH₄ and ˜50% of anthropogenic N₂O to livestock activities; estimates vary by methodology and year.⁵ In terms of land use, more than three-quarters of global agricultural land is dedicated to livestock-grazing land plus cropland used to grow animal feed-constraining terrestrial biodiversity and carbon storage potential.⁶ Accordingly, there is a need for alternative protein production systems that can reduce emissions and land requirements while meeting demand for meat products.

Cellular agriculture is an emerging field focused on producing agricultural products from cell cultures rather than whole animals. Cultured meat (also called cell-cultivated meat) is produced by proliferating animal cells in controlled environments and differentiating them into muscle and associated tissues suitable for consumption.⁷ In representative processes, stem or progenitor cells are expanded in nutrient media in bioreactors and can be seeded onto edible or biodegradable scaffolds that provide structure; process design addresses media formulation, oxygen and mass transfer, scale-up, and downstream harvesting.⁸ Advances in scaffold materials and fabrication (e.g., plant-derived scaffolds, microcarrier systems, aligned fiber matrices, and 3D bioprinting) aim to support myogenesis and achieve target textures for meat analogs while enabling cost-effective, scalable production. These approaches have potential to supplement conventional meat supplies and to reduce environmental burdens associated with livestock production.

### Method and System for Producing a Hybrid Cultured Meat Product

The disclosure provides a method and system for producing a hybrid cultured meat product that may incorporate seeded decellularized plant scaffolds, with a protein source and a binding agent.

Various plants, fruits, and vegetables can be used to make decellularized scaffolds. In certain embodiments, the plant scaffolds comprise plants, fruits, and vegetables selected from broccoli, cauliflower, mushrooms, spinach, celery, and apple. In certain embodiments, the plant scaffolds comprise plants, fruits, and vegetables selected from broccoli florets, spinach leaves, corn stover, jackfruit rinds, parsley leaves and stem, calathea, vanilla, anthurium, orchid, bamboo, solenostemon (coleus), Artemisia annua (aka sweet wormwood) leaves, peanut hairy roots, seaweed, rockweed, apple peels, banana peels, orange peels, and/or blades of grass. In certain embodiments, the plant scaffolds comprise broccoli, cauliflower, mushrooms, spinach, celery, and apple. In certain embodiments, the plant scaffolds comprise broccoli florets.

The decellularized plant scaffolds can be seeded with mammalian or poultry cells. Numerous types of cells are suitable for this purpose including, but not limited to, (i) primary skeletal muscle satellite cells or myoblasts from bovine, porcine, ovine, and/or poultry; (ii) adipogenic precursors: preadipocytes or adipose-derived stromal/stem cells (ADSCs) for lipid deposition and flavor; (iii) mesenchymal stromal/stem cells (MSCs): multipotent progenitors that can be directed to myogenic, adipogenic, or fibrogenic lineages; (iv) fibroblasts and connective-tissue progenitors: to tune texture via extracellular matrix deposition; (v) endothelial or endothelial-like cells: optional for co-culture support or perfusability studies; (vi) induced pluripotent stem cell (iPSC)-derived myogenic or adipogenic progenitors: scalable sources for manufacturing; and (vii) co-cultures (e.g., myogenic+adipogenic±fibroblasts/endothelial) to approximate whole-muscle composition. In some embodiments, the decellularized plant scaffolds is seeded with QM7 cells, a quail myoblast line that was originally established from the pectoral muscle of a Japanese quail embryo.

The protein source is derived from one or more of peas, soybeans, wheat, chickpeas, lentils, and mycoprotein, or a combination thereof. In some embodiments, the protein source comprises pea protein. This protein source is in addition to the proteins derived from the above-described mammalian or poultry cells seeding the decellularized plant scaffolds.

The binding agent comprises can be selected from one or more of transglutaminase, alginate+Ca²⁺ (0.5-2.0%), gellan gum (0.1-0.6%), K-carrageenan (works well with pea/soy matrices at ˜0.5-2%), methylcellulose (0.5-3.0%), low-methoxyl pectin+Ca²⁺ (0.5-2.0% pectin; 5-30 mM Ca²⁺), konjac glucomannan+xanthan (0.2-1.0% KGM+0.05-0.2% xanthan), laccase+pectin (50-500 U/kg laccase; 0.2-1% pectin), tyrosinase (polyphenol oxidase; 20-200 U/kg), wheat gluten (3-8%), sodium caseinate (0.5-2.0%), egg albumen (1-3%), and plant fiber concentrates (e.g., citrus/apple/pea; 0.5-3.0%). In some embodiments, the binding agent comprises transglutaminase.

The process may involve decellularizing the plant scaffolds through sequential exposure to agents like sodium dodecyl sulfate (SDS) and bleach, followed by rinsing to achieve a low residual DNA level. The decellularized scaffolds can be mixed with pea protein and transglutaminase to form a mixture, which may be processed on an incubator-shaker to enhance the binding and texture. The mixture can be dried to obtain a moldable mass, which may be shaped into the desired form of the hybrid cultured meat product. This approach may eliminate the need for a cell harvest step and can create a product with unique textures and nutritional values derived from both the plant and protein components. The system may include containers and a rinsing apparatus to facilitate the decellularization process, ensuring the preparation of plant scaffolds for use in the cultured meat composition.

In the context of producing a hybrid cultured meat product, the process may involve the mixing of seeded decellularized plant scaffolds with a protein source and a binding agent to create a mixture. This mixture may then be formed into a desired shape to create the hybrid cultured meat product. In some embodiments, the decellularized plant scaffolds, which may include decellularized broccoli floret scaffolds and may be seeded with QM7 cells, can be combined with pea protein and transglutaminase. The mixture may be formed from these components, and the mass may be shaped into a desired form. The decellularized broccoli floret scaffold, pea protein, and transglutaminase may be integral to forming the cultured meat composition. The process may involve the incorporation of decellularized plants, which may be bound together to create unique textures and marketable products. The decellularized plant scaffolds may provide different textural and nutritional values, enhancing the final product characteristics. The binding agent, such as transglutaminase, may be added to the mixture before and after drying to enhance binding. The mixture may be processed on an incubator-shaker to facilitate the formation of the cultured meat. The drying of the mixture by freeze-drying and optionally air-drying may result in a moldable mass, which can then be kneaded with the binding agent to integrate it fully. The final shaping of the mass may result in the creation of the hybrid cultured meat product, which may possess unique textures and nutritional values derived from the combination of the decellularized plant scaffolds and the protein source.

The process of decellularizing plant scaffolds may involve sequential exposure to decellularizing agents, such as sodium dodecyl sulfate (SDS) and bleach, followed by rinsing. This procedure may be designed to prepare the plant scaffolds for subsequent use in creating a cultured meat composition. The decellularization may be confirmed by CYQUANT® to ensure that the residual DNA level is at or below a specified threshold, which may be important for the integrity of the final product. The seeded decellularized broccoli floret scaffold may then be incorporated into a mixture, which may include other components such as pea protein and transglutaminase. This mixture may be formed into a desired shape, potentially resulting in a hybrid cultured meat product. The seeded decellularized plant scaffolds may be bound together via cells, which may provide unique textures and nutritional values to the final product. The incorporation of decellularized plants into cultured meat applications may enhance the product's marketability by offering different textural and nutritional characteristics. The process may also involve the use of containers for holding the decellularizing agents and the plant scaffolds, as well as a rinsing apparatus for removing the decellularizing agents. The addition of transglutaminase to the mixture before and after drying may enhance the binding of the components, facilitating the formation of the cultured meat composition. The entire process may be designed to create a unique and marketable product by leveraging the properties of decellularized plant scaffolds and other components.

The mixture may be processed on an incubator-shaker at approximately 100 rpm and around 37° C. for a duration of about 24 hours. This processing step may facilitate the formation of the cultured meat by ensuring the mixture is adequately incubated. The incubator-shaker may provide a controlled environment that can enhance the interaction between the components of the mixture, potentially leading to a more uniform and stable product. The processing on the incubator-shaker may be significant in achieving the desired consistency and texture of the cultured meat product. The mixture, once processed, may be further subjected to additional steps to complete the formation of the hybrid cultured meat product.

The process may begin with the drying of the mixture, which can be achieved through freeze-drying for a period ranging from 24 to 72 hours, and optionally, air-drying for a duration of 24 to 120 hours. This drying step may be necessary to obtain a moldable mass, which can then be prepared for subsequent shaping. The drying process may involve the removal of moisture from the mixture, potentially resulting in a mass that can be easily manipulated into a desired form. The moldable mass, once obtained, may serve as a precursor for the final product, allowing for the integration of additional components or the application of further processing techniques. The drying of the mixture may be a step in ensuring the stability and integrity of the moldable mass, which can then be utilized in the formation of the hybrid cultured meat product. The potential for achieving a moldable mass through this drying process may provide flexibility in the production of various shapes and forms, catering to diverse consumer preferences and market demands.

The process may involve kneading the binding agent into the moldable mass, which may facilitate the integration of the binding agent. This action may be correlated with the step of forming the mass into a desired shape, which may be intended to shape the final product. The binding agent, which may be transglutaminase, may be kneaded into the moldable mass to enhance the structural integrity and cohesiveness of the final product. The moldable mass, which may be obtained through drying processes, may be shaped into a desired form, potentially allowing for the creation of a hybrid cultured meat product with specific textures and appearances. The integration of the binding agent into the moldable mass may be important for achieving the desired mechanical properties and stability of the final product. The shaping of the mass may be a step in the production process, as it may determine the final form and presentation of the hybrid cultured meat product. The actions of kneading and forming may be interconnected, as the successful integration of the binding agent may directly influence the ability to shape the mass effectively. The process may be designed to ensure that the binding agent is evenly distributed throughout the moldable mass, which may contribute to the uniformity and quality of the final product. The potential for creating unique and marketable products may be enhanced by the ability to shape the moldable mass into various forms, which may appeal to consumer preferences and market demands.

In the context of producing a hybrid cultured meat product, containers may be utilized for holding decellularizing agents and plant scaffolds, while a rinsing apparatus may be employed to remove the decellularizing agents. This process may facilitate the decellularization of plant scaffolds, which can be important for preparing the scaffolds for subsequent use. Transglutaminase may be added to the mixture both before and after drying, which may enhance the binding of the components within the mixture. The mixture may comprise decellularized broccoli floret scaffold, pea protein, and transglutaminase, which may form a cultured meat composition. The inclusion of transglutaminase may serve to integrate the binding agent effectively, potentially contributing to the structural integrity of the final product. The decellularized broccoli floret scaffold, in combination with pea protein and transglutaminase, may form a cohesive mixture that can be shaped into a desired form, thereby creating the hybrid cultured meat product. The process may allow for the creation of unique textures and nutritional values, which may enhance the marketability and appeal of the final product.

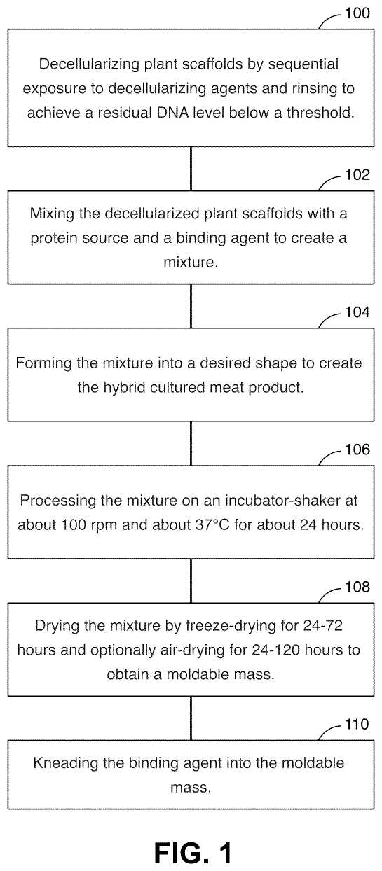

FIG. 1 is a flowchart illustrating a method in step 100 for decellularizing plant scaffolds by sequential exposure to decellularizing agents and rinsing to achieve a residual DNA level below a threshold, according to an embodiment. At step 100, the plant scaffolds, which may comprise broccoli florets, can be subjected to a decellularization process involving sequential exposure to decellularizing agents such as sodium dodecyl sulfate (SDS) and bleach. This process may involve two SDS (5% w/v) treatments over two days and a bleach (5% w/v) treatment over twenty-four hours. The decellularization process may aim to achieve a residual DNA level at or below 0.01 μg/mL or at or below 2% of fresh plant scaffolds. The decellularized plant scaffolds may then be prepared for use in creating a hybrid cultured meat product. The decellularization process may be confirmed by CYQUANT® to ensure that the residual DNA level is at or below the specified threshold. The decellularized broccoli floret scaffold may be seeded with QM7 cells. The seeded decellularized broccoli floret scaffold may then be incorporated into a mixture comprising pea protein and transglutaminase to form a cultured meat composition. This composition may be formed from a mixture that includes decellularized florets, pea protein, and transglutaminase added before and after drying. The incorporation of decellularized plants into cultured meat applications may provide unique textures and nutritional values to the final product.

In step 102, the process may involve the mixing of seeded decellularized plant scaffolds with a protein source and a binding agent to create a mixture. The decellularized plant scaffolds may be derived from broccoli florets, which have undergone a decellularization process involving sodium dodecyl sulfate (SDS) and bleach followed by seeding with QM7 cells. The protein source in this context may comprise pea protein, which is integrated into the mixture to provide nutritional value and structural support. The binding agent, identified as transglutaminase, may be added to the mixture both before and after the drying process to enhance the binding properties and ensure the structural integrity of the final product. The mixture may be composed of approximately 1.00 g (wet) decellularized plant scaffolds, about 0.25 g of the protein source, and about 0.10 g of the binding agent added before drying. An additional 0.10 g of the binding agent may be incorporated after the drying process to further reinforce the mixture. This step may be important in forming a cohesive and moldable mass that can be shaped into the desired form of the hybrid cultured meat product. The integration of these components may allow for the creation of a product that combines the textural and nutritional benefits of both plant and protein sources, potentially resulting in a unique and marketable hybrid cultured meat product.

At step 104, the mixture may be formed into a desired shape to create the hybrid cultured meat product. The mixture, which may comprise decellularized plant scaffolds, a protein source, and a binding agent, can be manipulated to achieve a specific form that is suitable for the final product. The process of shaping the mixture may involve techniques that allow for the integration of the various components, ensuring that the decellularized plant scaffolds, such as broccoli florets, are effectively combined with the protein source, like pea protein, and the binding agent, transglutaminase. This shaping process may be important in determining the texture and structural integrity of the hybrid cultured meat product. The potential for creating unique textures and nutritional profiles may be enhanced by the careful selection and combination of these components. The resulting product may exhibit characteristics that are distinct from traditional meat products, offering a novel alternative in the food industry. The formation of the mixture into a desired shape may also facilitate the subsequent processing steps, such as drying or further modification, to achieve the final product specifications.

At step 106, the mixture may be processed on an incubator-shaker at approximately 100 rpm and around 37° C. for a duration of about 24 hours. This processing step may serve to incubate the mixture, potentially facilitating the integration of its components. The mixture, which may comprise decellularized broccoli floret scaffold, pea protein, and transglutaminase, may undergo a transformation during this incubation period. The incubator-shaker may provide a controlled environment that can enhance the interaction between the protein source and the binding agent, possibly leading to a more cohesive mixture. The processing conditions, including the rotational speed and temperature, may be selected to optimize the biochemical reactions within the mixture, potentially resulting in a product with desirable textural and nutritional properties. The use of an incubator-shaker may also ensure uniform exposure of the mixture to the set conditions, which can be important for achieving consistent quality in the final cultured meat product. This step may be integral to the overall method, as it may lay the groundwork for subsequent processing stages that further refine the mixture into a moldable mass and eventually into the desired shape of the hybrid cultured meat product.

In step 108, the process of drying the mixture may be initiated to obtain a moldable mass. The mixture, which may include seeded decellularized plant scaffolds, a protein source, and a binding agent, can undergo freeze-drying for a period ranging from 24 to 72 hours. This step may be followed by optional air-drying for an additional 24 to 120 hours. The drying process may serve to remove moisture from the mixture, thereby transforming it into a moldable mass that can be further processed. The freeze-drying technique may involve sublimation, where the frozen water content in the mixture is directly converted into vapor, potentially preserving the structural integrity and nutritional properties of the components. The optional air-drying phase may further reduce the moisture content, enhancing the moldability of the mass. This step may be important in preparing the mixture for subsequent shaping into the desired form of the hybrid cultured meat product. The moldable mass obtained from this drying process may exhibit unique textural properties, which can be attributed to the combination of the decellularized plant scaffolds and the protein source. The drying step may also facilitate the integration of the binding agent, ensuring that the final product maintains its structural cohesion. The potential for achieving a moldable mass through this drying process may be integral to the overall method of producing a hybrid cultured meat product, as it allows for the creation of a product with specific textures and nutritional values.

In the context of step 110, the process may involve kneading the binding agent into the moldable mass. This step may be important in ensuring that the binding agent, identified as transglutaminase, is thoroughly integrated into the moldable mass, which may consist of a mixture of decellularized plant scaffolds, a protein source, and the binding agent itself. The kneading action may facilitate the uniform distribution of the transglutaminase throughout the moldable mass, potentially enhancing the structural integrity and cohesiveness of the final hybrid cultured meat product. The moldable mass, having been previously dried to achieve a suitable consistency, may be prepared to receive the binding agent in a manner that allows for optimal interaction between the components. This interaction may be essential for achieving the desired textural properties and ensuring that the final product maintains its shape and form. The integration of the binding agent through kneading may also contribute to the unique textures and nutritional values that are characteristic of the hybrid cultured meat product. The process may be designed to accommodate variations in the composition of the moldable mass, allowing for the creation of diverse and marketable products with distinct textural and nutritional profiles.

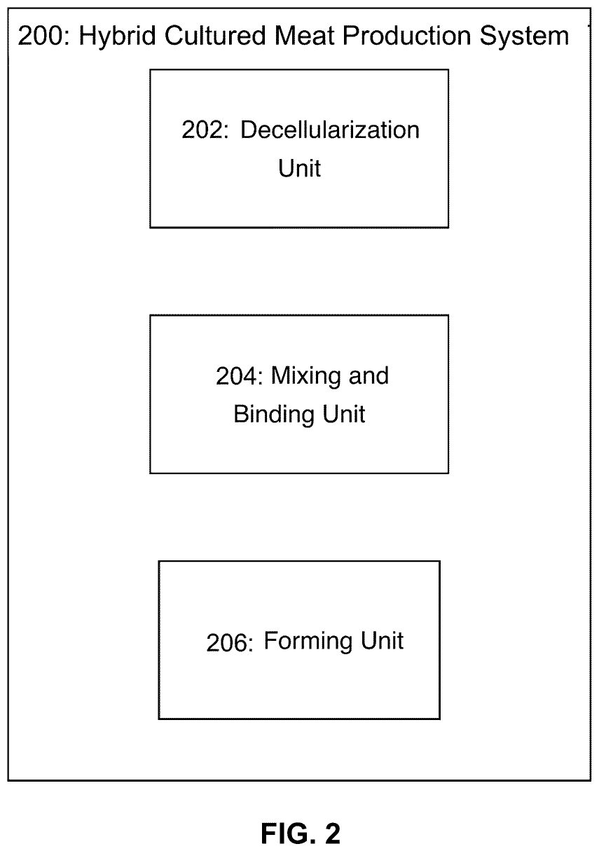

FIG. 2 illustrates an embodiment of a Hybrid Cultured Meat Production System. The Decellularization Unit, identified as component 202, may be responsible for the decellularization of plant scaffolds. This process may involve the sequential exposure of plant scaffolds to decellularizing agents, such as sodium dodecyl sulfate (SDS) and bleach, followed by rinsing. The decellularization may be confirmed by CYQUANT® to ensure that the residual DNA level is at or below a specified threshold. The Decellularization Unit 202 may utilize containers to hold the decellularizing agents and the plant scaffolds, along with a rinsing apparatus to effectively remove the decellularizing agents. The decellularized plant scaffolds, such as broccoli floret scaffolds, may then be prepared for further processing. The decellularized scaffolds may seeded with QM7 cells and then be mixed with a protein source and a binding agent to create a mixture, which may be processed on an incubator-shaker to facilitate the formation of a cultured meat composition. The mixture may undergo drying, potentially through freeze-drying and air-drying, to obtain a moldable mass. This mass may be kneaded with a binding agent, such as transglutaminase, to enhance binding and facilitate the formation of the final product. The Decellularization Unit may play a role in preparing the plant scaffolds for use in creating a hybrid cultured meat product, which may offer unique textures and nutritional values derived from the combination of decellularized plant scaffolds and protein sources.

The Mixing and Binding Unit, identified as component 204, may serve a role in the production of hybrid cultured meat products. This unit may be responsible for the integration of seeded decellularized plant scaffolds with a protein source and a binding agent to form a cohesive mixture. The seeded decellularized plant scaffolds, which may include components such as pea protein and transglutaminase, may be mixed to create a uniform mixture. The process may involve the careful addition of transglutaminase both before and after the drying phase to ensure optimal binding within the mixture. The mixture may then be processed on an incubator-shaker, which may operate at approximately 100 rpm and 37° C. for a duration of about 24 hours, to facilitate the integration of the components. This processing step may be important for achieving the desired consistency and texture of the mixture. Following this, the mixture may undergo a drying process, which may include freeze-drying for a period of 24-72 hours and optionally air-drying for 24-120 hours, to yield a moldable mass. The moldable mass may then be kneaded with additional transglutaminase to enhance the binding properties before being formed into the desired shape. This sequence of steps may ensure that the final product possesses the unique textures and nutritional values derived from the combination of the decellularized plant scaffolds and the protein source. The Mixing and Binding Unit 204 may thus play a role in the creation of a hybrid cultured meat product by facilitating the thorough integration and binding of its constituent components.

The Forming Unit, identified as component 206, may be responsible for shaping the mixture into the desired form to create the final hybrid cultured meat product. This process may involve the integration of decellularized broccoli floret scaffolds seeded with QM7 cells, pea protein, and transglutaminase, which are mixed to form a cultured meat composition. The forming action may be facilitated by the mixture being processed on an incubator-shaker, which may ensure uniformity and consistency in the final product. The mixture may then undergo a drying process, potentially involving freeze-drying and air-drying, to achieve a moldable mass. This moldable mass may be further processed by kneading additional transglutaminase into it, which may enhance the binding properties and structural integrity of the final product. The Forming Unit 206 may then shape this mass into the desired configuration, which may be important for the final presentation and marketability of the hybrid cultured meat product. Possible configurations include, but are not limited to, a spherical meatball shape, a sausage patty shape, a sausage shape, a hamburger patty shape, a chicken nugget shape. The entire process may be designed to ensure that the final product retains unique textures and nutritional values derived from the combination of plant scaffolds and protein sources.

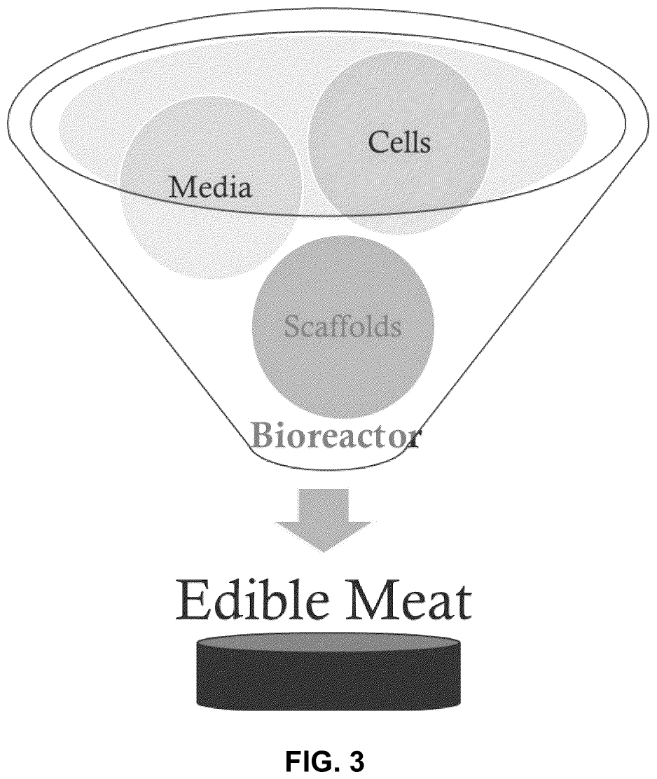

FIG. 3 provides a conceptual overview of a system for producing cultured meat. The diagram shows that key inputs, including Scaffolds, Cells, and Media are combined within a Bioreactor. The process results in the creation of Edible Meat. This figure illustrates the general context in which the specific inventive method, utilizing decellularized plant scaffolds, operates.

Some embodiments of the technology described herein can be defined according to any of the following numbered paragraphs:
1. A cultured meat composition comprising: (a) a decellularized broccoli floret scaffold seeded with QM7 cells; (b) pea protein; and (c) transglutaminase; wherein the broccoli florets are decellularized by sequential exposure to sodium dodecyl sulfate (SDS) and bleach comprising two SDS (5% w/v) treatments over two days and a bleach (5% w/v) treatment over twenty-four hours, followed by rinsing; and wherein the cultured meat is formed from a mixture comprising 1.00 g (wet) decellularized florets, about 0.25 g pea protein, about 0.10 g transglutaminase added before drying, and about 0.10 g transglutaminase added after drying.
2. The composition of paragraph 1, wherein residual DNA after decellularization is at or below 0.01 μg/mL or at or below 2% of fresh as determined by a CYQUANT® assay.
3. The composition of paragraph 1 or 2, further comprising beetroot extract at about 0.015 g per g of decellularized florets.
4. The composition of any of paragraphs 1-3, wherein the mixture is processed on an incubator-shaker at about 100 rpm and about 37° C. for about 24 hours before drying.
5. The composition of any of paragraphs 1-4, wherein drying comprises freeze-drying for 24-72 hours and optionally air-drying for 24-120 hours.
6. The composition of any of paragraphs 1-5, wherein oven-drying is excluded.
7. The composition of any of paragraphs 1-6, wherein the cultured meat is in the shape of a meatball having a diameter of 20-30 mm.
8. The composition of any of paragraphs 1-7, wherein the pea protein comprises pea-protein isolate and/or concentrate.
9. A method of making a cultured meat composition comprising: (a) decellularizing broccoli florets by sequentially exposing the florets to SDS comprising two SDS (5% w/v) treatments over two days and a bleach (5% w/v) treatment over twenty-four hours, and rinsing; (b) confirming decellularization by CYQUANT® to provide a residual DNA level at or below 0.01 μg/mL or at or below 2% of fresh; (c) seeding the decellularizing broccoli florets with QM7 cells; (d) mixing 1.00 g (wet) of the seeded decellularized florets with about 0.25 g pea protein and about 0.10 g transglutaminase and processing the mixture on an incubator-shaker at about 100 rpm and about 37° C. for about 24 hours; (e) drying the mixture by freeze-drying for 24-72 hours and optionally air-drying for 24-120 hours to obtain a moldable mass; (f) kneading about 0.10 g transglutaminase into the mass; and (g) forming the mass into a desired shape.
10. The method of paragraph 9, further comprising adding beetroot extract at about 0.015 g per g of decellularized florets prior to forming.
11. The method of paragraph 9 or 10, further comprising removing interstitial water by centrifugation and aspiration prior to drying.
12. The method of any of paragraphs 9-11, wherein oven-drying is excluded.
13. The method of any of paragraphs 9-12, wherein the desired shape is a spherical meatball having a diameter of 20-30 mm.
14. The composition of paragraph 1, further comprising cultured animal cells adhered within the decellularized florets.
15. The method of paragraph 9, further comprising contacting the decellularized florets or the formed cultured meat composition with a cell suspension at a concentration of 1.5×10⁶ to 3.0×10⁶ cells/mL for 1-4 hours to allow adhesion without surface coatings.
16. The method of paragraph 15, wherein adhesion time is about 1 hour at a concentration of about 3.0×10⁶ cells/mL.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Example 1 Scaffold Preparation

Various plants, fruits, and vegetables can be used to make decellularized scaffolds. These include, but are not limited to, broccoli florets, spinach leaves, corn stover, jackfruit rinds, parsley leaves and stem, calathea, vanilla, anthurium, orchid, bamboo, solenostemon (coleus), Artemisia annua (aka sweet wormwood) leaves, peanut hairy roots, seaweed, rockweed, apple peels, banana peels, orange peels, and blades of grass.

Generally, the plant tissue can be decellularized using any methods known in the art for decellularizing tissue.

In one embodiment, the plant tissue is decellularized via detergent perfusion using at least one of a detergent and enzyme. Exemplary perfusion methods include immersion in detergents and bleaching agents such as sodium hypochlorite (bleach), sodium dodecyl sulfate (SDS), ethylenediaminetetraacetic acid (EDTA), Triton X-100, and the like, and combinations thereof. Exemplary enzymes for use in decellularization include lipases, thermolysin, galactosidases, nucleases (e.g., endonucleases such as benzoase), trypsin, and combinations thereof. In some embodiments, the plant tissue can be decellularized using a mixture of detergent and enzyme, such as a mixture of EDTA and trypsin.⁹

In another embodiment, prior to the perfusion of different detergent solutions, the plant tissues were treated to remove their cuticles through three one-minute serial washes with hexanes (98%, Mixed Isomers, Alfa Aesar, Haverhill, MA) and 1× PBS. 1% SDS in deionized water was perfused through the leaf for 1 day, followed by another 1 day perfusion of 0.1% TritonX-100 in 10% bleach and followed by 1 day perfusion of deionized water. After decellularization was complete, leaves were washed in 10 mM, pH 9.0 tris-HCl buffer (Sigma Aldrich, St. Louis, MO) for 12 h. The leaves were then frozen overnight prior to being lyophilized for 24 h, then stored at room temperature. Prior to any investigation, lyophilized leaves were rehydrated in the same tris buffer solution for at least 3 h before being washed twice with 1× PBS.¹⁰

In addition to perfusion of detergents, plant tissues were also decellularized by submerging the samples into the solutions, under constant mixing. The solutions are similar to the perfusion method. The overall sequence is SDS solution (detergent 1) followed by a solution composed of a second detergent (Triton X-100 or Polysorbate 20) with bleach. All protocols can be preceded by a hexane treatment to remove cuticles.

The study first assessed various decellularization protocols. The solution components and reaction times were evaluated to identify the conditions that were most reproducible, shorter and best adapted to specific plants. The concentrations investigated were SDS (1-10%), Polysorbate 20 (1-10%) and Bleach (1-10%). Reaction times were evaluated from 1 to 7 days. Results were confirmed using CYQUANT® or image analysis. The following protocols in Table 1 were identified and are the ones currently used in our studies:

TABLE 1
Decellularization Protocols

| Protocol | Solution 1 | Solution 2 | Solution 3 |
|---|---|---|---|
| 1 | SDS (1%), 5 days | Tween-20 (1%) + | DI H₂O, |
| | | Bleach (3%), 2 days | 1 day/storage |
| 2 | SDS (5%), 2 days | Bleach (5%), 2 days | DI H₂O, |
| | | | 1 day/storage |

| | | |
|---|---|---|
| 3 | SDS (10%) + Polysorbate 20 (3%) + | DI H₂O, |
| | Bleach (10%), 1 day | 1 day/storage |

Abbreviations/Sources: Deionized water (DI H₂O); Bleach (The Clorox Co., Oakland, CA, USA); Sodium Dodecyl Sulfate (SDS) (Sigma-Aldrich, St. Louis, MO, USA); Polysorbate 20 (Tween-20) (Sigma-Aldrich).

Decellularization Protocol 1 is the most commonly used and is applied to spinach leaves. Decellularization Protocol 2 is mainly used for broccoli florets (the scaffolds used in the Examples below). Decellularization Protocol 3 is used for rapid decellularization if time constraints require it.

### Example 2 Decellularization of Broccoli Florets

Fresh organic broccoli was purchased at a local marketplace. Florets were detached from the broccoli stalk using a scalpel blade, ensuring that each carrier consisted of a single bulb. Samples were rinsed thoroughly with distilled water.

One gram of florets was placed in a 50 mL conical tube containing 45 mL of deionized water (DI H₂O) with 5% SDS (Sigma-Aldrich, St. Louis, MO, USA) and gently agitated on a laboratory roller for 48 h. The SDS decellularization solution was aspirated and replaced after 24 h. Next, the SDS decellularization solution was aspirated from the samples and replaced by a solution of 5% bleach (The Clorox Co., Oakland, CA, USA) and gently agitated on a laboratory roller for 24 h. Finally, the bleach decellularization solution was aspirated from the samples and the florets were then transferred to a 2 L beaker containing DI H₂O for at least 1 h, while replacing the DI H₂O every 15 min. The samples were stored in DI H₂O at room temperature.

### DNA Content

The total DNA content of fresh and decellularized scaffolds was measured using a CYQUANT® DNA assay kit (Thermo Fisher Scientific, Waltham, MA, USA) to confirm decellularization. To ensure comprehensive DNA release, samples were snap-frozen using liquid nitrogen and homogenized using iris scissors. The total DNA content was calculated by comparing the intensity of sample fluorescence with that of a standard curve at an excitation wavelength of 480 nm and an emission wavelength of 520 nm per the manufacturer's recommendation. Fluorescence intensity measurements were taken using a PerkinElmer Victor3 spectrophotometer (PerkinElmer, Waltham, MA, USA).

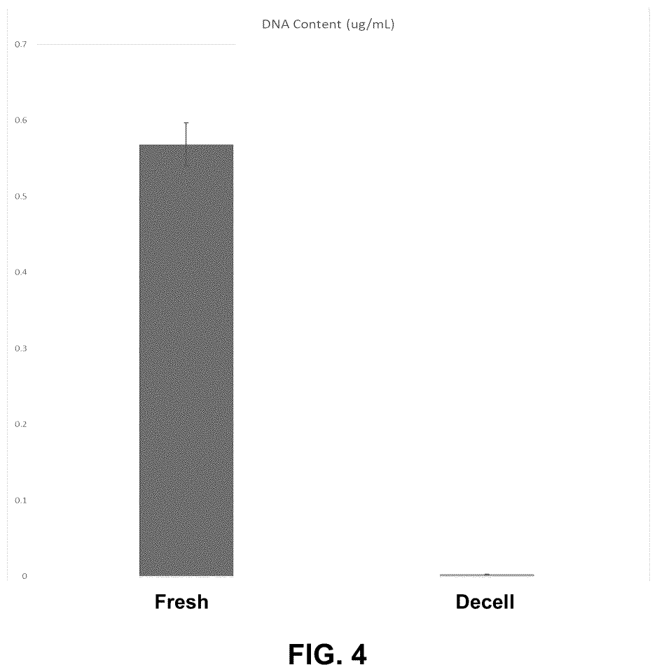

The average DNA content (μg/mL) in fresh broccoli florets (left bar) and the decellularized broccoli florets (right bar) is shown in FIG. 4. The average DNA content in fresh broccoli florets was 0.568 μg/mL compared to the average DNA content in decellularized broccoli florets which was 0.003 μg/mL, thus confirming the successful decellularization by Decellularization Protocol 2.

### Example 3 Seeding Density Experiments

Cell growth and health onto the decellularized broccoli florets is critical to create a nutritionally and flavor equivalent meat product. Initial cell seeding can affect the growth phase length and cell viability. The lower the number of cells loaded, the simpler it is to start the process. Therefore, different cell seeding methods were investigated to reduce the cell loadings, maximize cell adhesion and condense the growth phase.

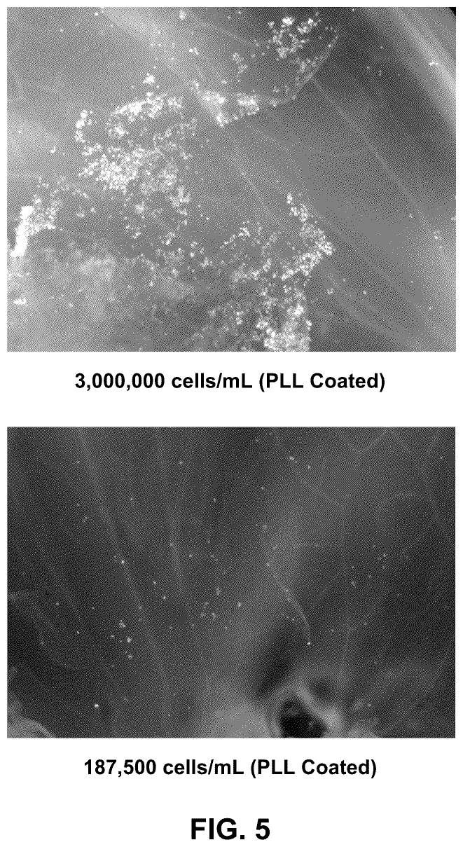

The variables analyzed were cell loading (cells/g decellularized florets), decellularized floret coating, and adhesion time. Cell loadings varied from 100,000 cells/g decellularized florets to 3 million cells/g. Seeding lasted 24 hours, then florets were fixed in PFA, stained with Hoechst, and imaged. The image analysis consisted of a quick visual cell count done on each floret, and the floret was placed into one of three categories: >100 cells; 30-100 cells; or <30 cells. FIG. 5 shows an image of PLL-coated florets loaded with the highest and lowest dose of cells.

Table 2 summarize the results of 24 hour adhesion of uncoated florets. The loading of 1.5 million/g florets was able to successfully seed (at least 30 cells per floret) in 78% of the florets. Accordingly, the loading of 1.5 million/g florets was used for further testing.

TABLE 2
Seeding Loading Results

| Cell loading (cells/g florets) | Adhered cells per floret | Rate (%) |
|---|---|---|
| 3,000,000 | >100 | 47 |
| | 30-100 | 28 |
| | <30 | 25 |
| 1,500,000 | >100 | 44 |
| | 30-100 | 34 |
| | <30 | 22 |
| 750,000 | >100 | 19 |
| | 30-100 | 41 |
| | <30 | 41 |
| 375,000 | >100 | 22 |
| | 30-100 | 22 |
| | <30 | 56 |
| 187,500 | >100 | 13 |
| | 30-100 | 34 |
| | <30 | 53 |

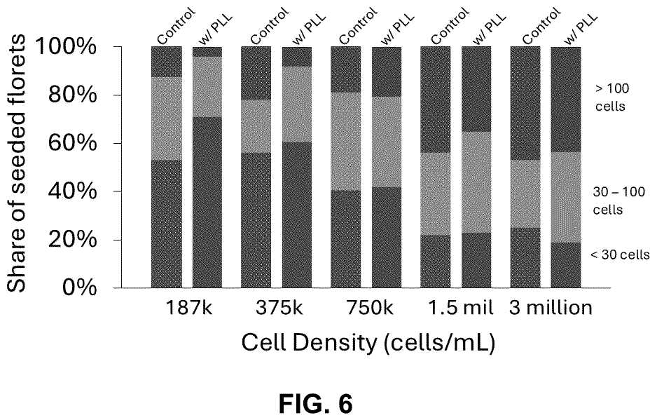

Four alternatives for floret coating were evaluated: poly-L-lysine (PLL), fibronectin, PLL+fibronectin, and gelatin. None of the coatings achieved higher adhesion than the florets by themselves after 24 hours. Adhesion time was gradually reduced from 24 hours to 4 hours without loss of seeding efficiency. When adhesion time was less than 4 hours, the seeding efficiency was reduced with 1.5 million cells/g. An increase in cell loading to 3 million cells/g achieved the 78% seeding efficiency after 1 hour adhesion time. See, FIG. 6 which provides a stacked bar chart presenting experimental data comparing the results for a ‘Control’ group and a ‘w/PLL’ group on cell seeding efficiency. The y-axis indicates the ‘Share of seeded florets,’ categorized by the number of cells attached (<30, 30-100, >100). The x-axis shows varying ‘Cell Density’ levels.

In the present study, QM7 cells were loaded on the decellularized broccoli florets. QM7 is a quail myoblast line widely used as a myogenic model. QM7 cells were originally established from the pectoral muscle of a Japanese quail embryo. However, numerous types of cells are suitable for this purpose including, but not limited to, (i) primary skeletal muscle satellite cells/myoblasts from bovine, porcine, ovine, and/or poultry; (ii) adipogenic precursors: preadipocytes or adipose-derived stromal/stem cells (ADSCs) for lipid deposition and flavor; (iii) mesenchymal stromal/stem cells (MSCs): multipotent progenitors that can be directed to myogenic, adipogenic, or fibrogenic lineages; (iv) fibroblasts and connective-tissue progenitors: to tune texture via extracellular matrix deposition; (v) endothelial(-like) cells: optional for co-culture support or perfusability studies; (vi) induced pluripotent stem cell (iPSC)-derived myogenic or adipogenic progenitors: scalable sources for manufacturing; and (vii) co-cultures (e.g., myogenic+adipogenic±fibroblasts/endothelial) to approximate whole-muscle composition. The selection can be guided by target product profile and culture conditions. Since the above data indicate that simple adhesion to the plant matrix is feasible without coatings, primary or progenitor cell types can be evaluated using similar seeding densities and contact times as demonstrated with QM7 cells.

### Example 4 Meatball Fabrication

The hybrid cultured meat product of the present disclosure can be formed in any shape or configuration. Possible configurations include, but are not limited to, a spherical meatball shape, a sausage patty shape, a sausage shape, a hamburger patty shape, a chicken nugget shape. In the present study, the spherical meatball shape was chosen.

The meatball fabrication is done using a) decellularized broccoli florets, b) food-grade Trans-glutaminase, c) pea protein and d) beetroot extract powder (food colorant). Trans-glutaminase acts is the binding agent for proteins and is already used for meat products. The optimum enzyme temperature is 50° C., although unstable above 40° C. The ideal temperature for cell growth is 37° C. In order to preserve long term enzyme activity and minimize damage to cells, the binding process is conducted at 37° C.

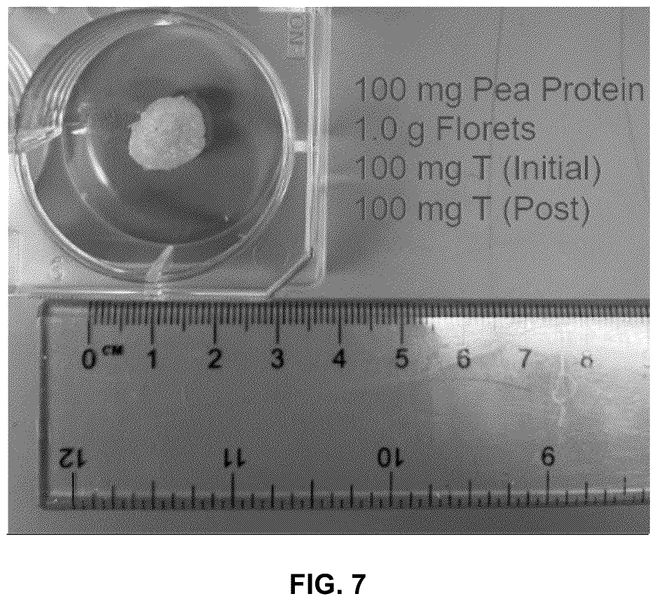

The development of the first version of the process involved varying Trans-glutaminase and pea protein loadings with 15-minute enzyme reactions. The loadings evaluated were for Trans-glutaminase (0.02-0.2 g protein/g decellularized florets) and for pea protein (0-0.5 g/g) all for overnight reaction. The lowest combination of added proteins that formed a moldable meatball was 0.1 g Trans-glutaminase/g decellularized florets and 0.1 g pea protein/g. A second addition of 0.1 g Trans-glutaminase/g decellularized florets prior to molding led to the formation of spherical bonded “meatballs” (see FIG. 7). This process resulted in Fabrication Protocol 1, which is summarized in Table 3.

TABLE 3
Hybrid Meatball Fabrication Protocol 1

| | |
|---|---|
| 1. | Weigh out seeded broccoli florets from storage jar, place them in a 15 |
| | mL conical tube |
| 2. | Add pea protein |
| 3. | Add transglutaminase |
| 4. | Add 3 mL DI Water to the conical tube |
| 5. | Place in water bath at 37° C. for ~15 min |
| 6. | Centrifuge for 3 minutes at 4000 RPM |
| 7. | Let stand overnight at room temperature |
| 8. | Next day, centrifuge for 3 minutes at 4000 RPM |
| 9. | Aspirate water from each tube |
| 10. | Let tubes sit at room temperature with caps off for 24 hours or until |
| | most of the water evaporates |
| 11. | Place solid contents of each tube into a 24 well plate |
| 12. | Weigh out second dosage of transglutaminase |
| 13. | With a scoopula, knead in the transglutaminase to each meatball for 2 |
| | minutes |
| 14. | With scoopula or your hands, form each meatball into a ball and |
| | place them in their respective wells |
| 15. | Leave the meatballs in the refrigerator (4° C.) for 48 hours |

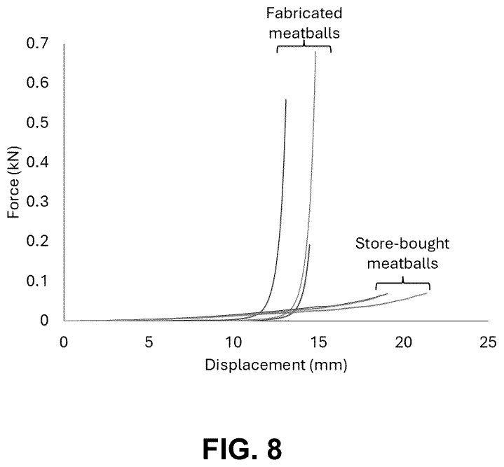

The process was then repeated at five times the mass (5 g decellularized florets) for five times larger meatballs. The drying portion was identified as critical to fabrication and air drying was not efficient. Three drying methods were evaluated: (i) oven drying, (ii) mechanical drying, and (iii) freeze drying. The oven dried meatballs went through Maillard reaction, changing the color and consistency of the meatballs which became brittle. Mechanical drying (squeezing and centrifugation) was not able to remove all the water. Freeze drying was done using a lyophilizer for 48 hours. Similarly, the meatballs were air dried for 72 hours to reach comparable dryness. These 5× meatballs mechanical properties were tested with a Instron testing system. The samples were compressed until completion at a rate of 5 mm/min. The different drying methods mechanical properties are summarized on Table 4. FIG. 8 shows the compression force over time for the fabricated and store-bought meatballs. The vertical y-axis represents the applied force in kilonewtons (kN), and the horizontal x-axis represents the displacement in millimeters (mm). The fabricated meatballs had approximately 15 mm diameter and the store-bought ones 25 mm. The inflection points are when the meatballs are almost completely compressed. The graph displays several data curves for ‘Fabricated meatballs’ (the product of the present disclosure) and ‘Store-bought meatballs’ (a comparative product). The steeper slope of the curves for the fabricated meatballs indicates a different textural profile, specifically a higher resistance to compression compared to the store-bought samples, which supports the creation of a product with unique textural values.

TABLE 4
Mechanical Properties of Different Drying Methods

| Drying method | Diameter | Compression stress |
|---|---|---|
| Air Drying | 15.00 ± 0.06 mm | 8.2 ± 0.7 MPa |
| Freeze drying | 15.31 ± 0.42 mm | 8.0 ± 1.2 MPa |
| Store bought frozen meatballs | 25.26 ± 2.65 mm | 0.13 ± 0.02 MPa |

When compared to a store-bought frozen meatball, the fabricated meatballs were not only too small, but the size difference led to statistically significant differences on compressive stress. These results indicated that freeze drying higher masses would be more efficient than air drying, resulting in the Fabrication Protocol 2, which is summarized in Table 5.

TABLE 5
Hybrid Meatball Fabrication Protocol 2

| | |
|---|---|
| 1. | Weigh out 6.0 g seeded decellularized broccoli florets from storage |
| | jar, place them in 50 mL conical tube |
| 2. | Add 1.2 g of pea protein |
| 3. | Add 1.2 g transglutaminase |
| 4. | Add 36 mL DI Water to the flask |
| 5. | Place in water bath at 37° C. for ~15 min |
| 6. | Centrifuge for 3 minutes at 4000 RPM |
| 7. | Let stand overnight at room temperature |
| 8. | Next day, centrifuge for 3 minutes at 4000 RPM |
| 9. | Aspirate water from each tube |
| 10. | Place tubes into freeze dryer for 48 hours |
| 11. | After drying, let tubes air dry for 96 hours |
| 12. | Place the solid contents of each tube into a 6-well plate |
| 13. | Weigh out second dosage of transglutaminase (1.2 g) |
| 14. | With a scoopula, knead in the transglutaminase to each meatball for 2 |
| | minutes |
| 15. | With scoopula or your hands, form each meatball into a ball and |
| | place them in their respective wells |
| 16. | Leave the meatballs in the refrigerator (4° C.) for 48 hours |

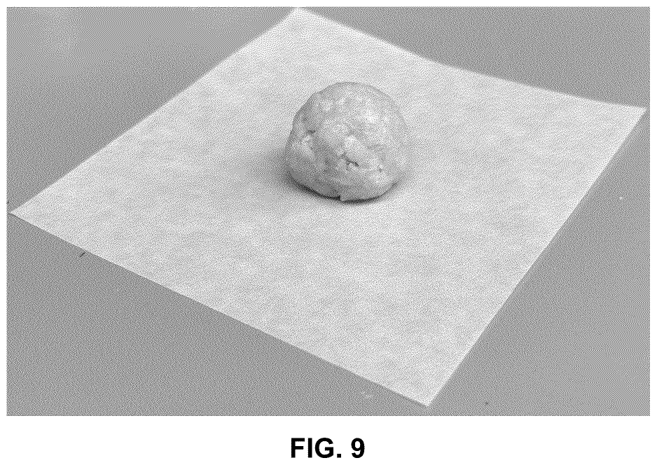

The enzymatic process for 10 times the initial loadings (10 g decellularized florets) were not successful. The binding was limited to a portion of the combined mass. Increased reaction time (from 15 minutes to 12 hours) did not improve binding. An incubator shaker was used to increase mixing and improve enzyme mass transfer and activity. The flasks were shaken at 100 rpm for 24 hours with the combination of protein, florets and trans-glutaminase. The same freeze-drying method as before was used. Meatballs of size equivalent to the store-bought frozen meatballs were successfully fabricated (24.3±0.6 mm, FIG. 9). The resulting method became the latest protocol, Fabrication Protocol 3, which is summarized in Table 6.

TABLE 6
Hybrid Meatball Fabrication Protocol 3

| | |
|---|---|
| 1. | Weigh out 6.0 g seeded decellularized broccoli florets from storage |
| | jar, place them a 50 mL Erlenmeyer flask |
| 2. | Weigh out 1.2 g of pea protein, place in the flask |
| 3. | Weigh out 1.2 g transglutaminase, place in the flask |
| 4. | Add 36 mL DI Water to the flask |
| 5. | Place in water bath at 37° C. for 15 min |
| 6. | Transfer the flask to an incubator shake. Incubate for 24 hours with |
| | agitation at 100 RPM at 37° C. |
| 7. | Aspirate water from each tube |
| 8. | Place tubes into freeze dryer for 48 hours |
| 9. | After drying, let tubes air dry for 96 hours |
| 10. | Place the solid contents of each tube into a petri dish |
| 11. | Add second dosage of transglutaminase (1.2 g) |
| 12. | With a scoopula, knead in the transglutaminase to each meatball for 2 |
| | minutes |
| 13. | With scoopula or your hands, form each meatball into a ball and |
| | place them in their respective wells |

### Example 5 Meatball Color Development and Analysis

As shown in FIG. 9, the fabricated meatballs were yellow in color. In order to achieve a raw meat color, organic beetroot extract was added to the meatballs. Organic Beetroot Extract Powder (Bulk Supplements.com) was used in the present study due to its high betalain content, which produced a dark red, meat-like, color.

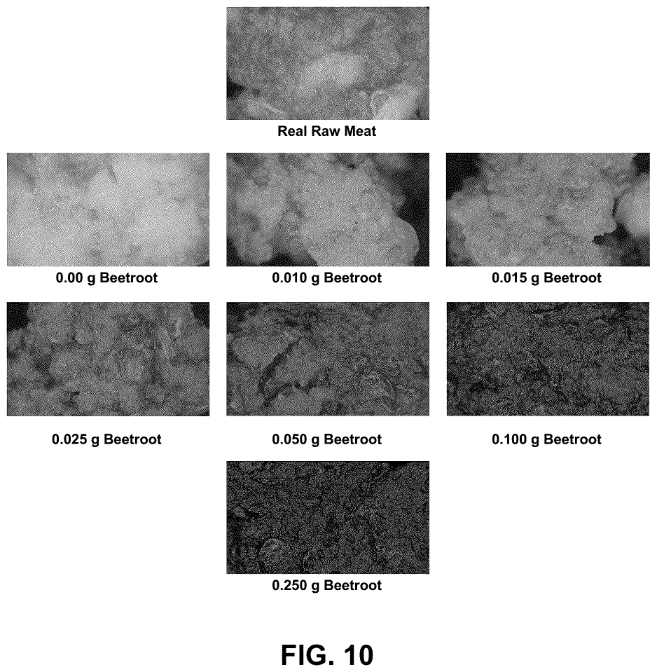

The extract loadings that were evaluated varied from 0-0.25 g/g decellularized florets. FIG. 10 displays a gallery of photographic images illustrating various embodiments of the fabricated hybrid cultured meat product with different concentrations of beetroot extract added for coloration, ranging from 0.000 g to 0.250 g. A sample of ‘Real Meat’ is included for comparison. The colors were analyzed by comparing the red, blue and green pixel values of the samples, as summarized in Table 4. The closest color to raw meat was achieved with 0.015 g beetroot extract/g decellularized florets.

TABLE 4
Color comparison of colored hybrid cultured
meat product with raw real meat

| Meatball | Red | Green | Blue |
|---|---|---|---|
| Raw meat | 190.9 | 101.5 | 92.0 |
| 0 g/g beetroot extract | 203.4 | 181.6 | 140.5 |
| 0.01 g/g beetroot extract | 198 | 138.1 | 125.9 |
| 0.015 g/g beetroot extract | 188.1 | 100.2 | 103.8 |
| 0.025 g/g beetroot extract | 152.2 | 78.3 | 83.6 |
| 0.05 g/g beetroot extract | 140.7 | 57.7 | 70.2 |
| 0.1 g/g beetroot extract | 109.9 | 50.2 | 61.3 |
| 0.25 g/g beetroot extract | 74.2 | 42.4 | 50.9 |

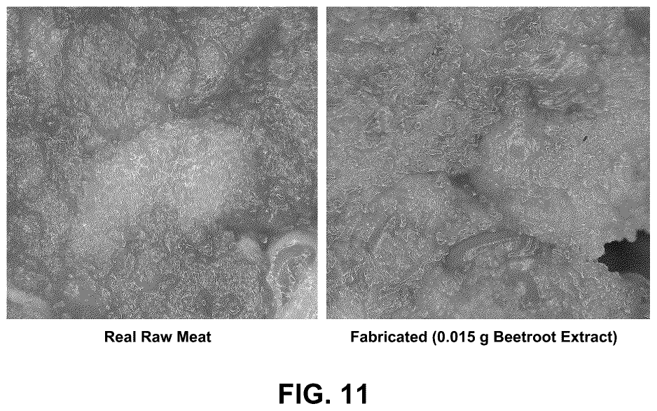

A closeup of the raw real meat (left) and the colored fabricated hybrid cultured meat with added beetroot extract (right) are shown in FIG. 11, which provides a side-by-side comparison allowing for a qualitative assessment of the visual similarity in color and texture between the product of the present disclosure and its conventional counterpart, demonstrating its potential as a meat alternative.

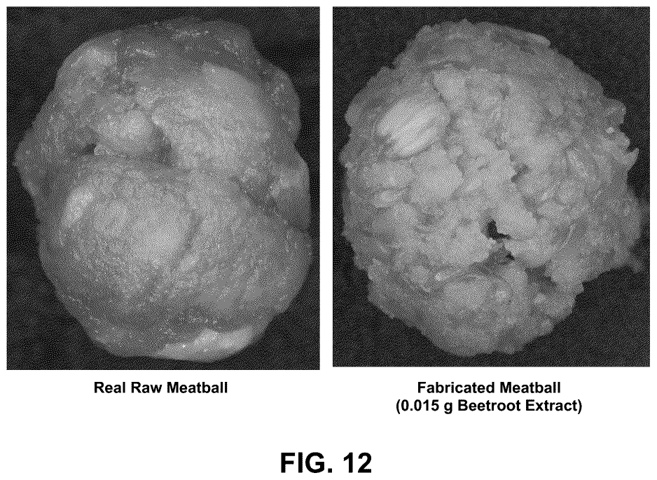

Finally, a direct comparison of raw uncooked meatball (left) and fabricated meatball with added beetroot extract (right) are shown in FIG. 12. This figure demonstrates the versatility of the product and process in creating unique marketable products.

### REFERENCES

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.
¹ See, e.g., Perreault, L. R., et al., (2023). “Repurposing agricultural waste as low-cost cultured meat scaffolds.” Front. Food. Sci. Technol. 3:1208298. doi: 10.3389/frfst.2023.1208298.
Thyden, R., et al., (2022). “An Edible, Decellularized Plant Derived Cell Carrier for Lab Grown Meat. Appl. Sci., 12:5155. https://doi.org/10.3390/app12105155.
Jones, J. D., et al., (2021). “Decellularized spinach: An edible scaffold for laboratory-grown meat.” Food Bioscience, 41:100986 Gershlak, J. R., et al., (2017). “Crossing Kingdoms: Using Decellularized Plants as Perfusable Tissue Engineering Scaffolds.” Biomaterials, 125:13-22. https://doi.org/10.1016/j.biomaterials.2017.02.011.
² United Nations, Department of Economic and Social Affairs, Population Division, World Population Prospects 2022 (Key Facts, 2022).
³ FAO, Tackling Climate Change Through Livestock: A global assessment of emissions and mitigation opportunities (2013).
⁴ FAO, Livestock and enteric methane (web resource, accessed Oct. 17, 2025): Agriculture contributes ˜40% of anthropogenic methane; livestock ˜32%.
⁵ Feedipedia (FAO/CIRAD/INRAE), Livestock and greenhouse gas emissions (citing IPCC/FAO analyses): Livestock ˜44% of anthropogenic CH₄ and ˜53% of anthropogenic N₂O (method- and year-dependent).
⁶ Our World in Data, Half of the world's habitable land is used for agriculture (updated 2024): >75% of agricultural land used for livestock (grazing+feed).
⁷ Stephens, N., et al., (2018). “Bringing cultured meat to market: Technical, socio-political, and regulatory challenges.” Trends in Food Science & Technology, 78:155-166.
⁸ Allan, S. J., et al., (2019). “Bioprocess Design Considerations for Cultured Meat Production with a Focus on Expansion Bioreactors.” Frontiers in Sustainable Food Systems 3:44.
Ben-Arye, T., et al., (2022). “Scaffolding technologies for the engineering of cultured meat: Towards the development of sustainable tissues for food.” Trends in Food Science & Technology, 129:58-72.
⁹See, e.g., PCT Published Application No. WO 2017/160862, “Functionalization of Plant Tissues for Human Cell Expansion.” Published: Sep. 21, 2017.
¹⁰ Gershlak, J. R., et al., (2025). “Biocompatibility of decellularized spinach leaves.” npj Biomed. Innov. 2:23. https://doi.org/10.1038/s44385-025-00028-8.

What is claimed is:

1. A method of producing a hybrid cultured meat product, the method comprising:
(a) decellularizing plant scaffolds by sequential exposure to decellularizing agents and rinsing to achieve a residual DNA level below a threshold;
(b) seeding the decellularizing plant scaffolds with cells;
(c) mixing the seeded decellularized plant scaffolds with a protein source and a binding agent to create a mixture; and
(d) forming the mixture into a desired shape to create the hybrid cultured meat product.

2. The method of claim 1, wherein the plant scaffolds comprise decellularized broccoli florets.

3. The method of claim 1, wherein the decellularizing agents comprise sodium dodecyl sulfate (SDS) and bleach.

4. The method of claim 3, wherein the decellularizing comprises two SDS (5% w/v) treatments over two days and a bleach (5% w/v) treatment over twenty-four hours.

5. The method of claim 1, wherein the residual DNA level is at or below 0.01 μg/mL or at or below 2% of fresh plant scaffolds.

6. The method of claim 1, wherein the decellularizing plant scaffolds are seeded with QM7 cells.

7. The method of claim 1, wherein the protein source comprises pea protein.

8. The method of claim 1, wherein the binding agent comprises transglutaminase.

9. The method of claim 8, wherein the transglutaminase is added to the mixture before and after drying.

10. The method of claim 1, wherein the mixture comprises about 1.00 g (wet) seeded decellularized plant scaffolds, about 0.25 g protein source, and about 0.10 g binding agent added before drying.

11. The method of claim 10, wherein about 0.10 g binding agent is added after drying.

12. The method of claim 1, further comprising processing the mixture on an incubator-shaker at about 100 rpm and about 37° C. for about 24 hours.

13. The method of claim 1, further comprising drying the mixture by freeze-drying for 24-72 hours and optionally air-drying for 24-120 hours to obtain a moldable mass.

14. The method of claim 13, further comprising kneading the binding agent into the moldable mass.

15. A hybrid cultured meat product produced by the method of claim 1.

16. The hybrid cultured meat product of claim 15, wherein the decellularized plant scaffolds are derived from one or more of broccoli, cauliflower, mushrooms, spinach, celery, and apple.

17. The hybrid cultured meat product of claim 15, wherein the decellularizing plant scaffolds are seeded with primary skeletal muscle satellite cells or myoblasts from bovine, porcine, ovine, or poultry.

18. The hybrid cultured meat product of claim 15, wherein the decellularizing plant scaffolds are seeded with QM7 cells.

19. The hybrid cultured meat product of claim 15, wherein the protein source is derived from one or more of peas, soybeans, wheat, chickpeas, lentils, and mycoprotein.

20. The hybrid cultured meat product of claim 15, wherein the binding agent comprises transglutaminase.

21. The hybrid cultured meat product of claim 15, wherein the hybrid cultured meat product comprises unique textures and nutritional values derived from the combination of the seeded decellularized plant scaffolds and the protein source.

22. A system for producing a hybrid cultured meat product, the system comprising:
(a) means for decellularizing plant scaffolds by sequential exposure to decellularizing agents and rinsing to achieve a residual DNA level below a threshold;
(b) means for seeding the decellularizing plant scaffolds;
(c) means for mixing the decellularized plant scaffolds with a protein source and a binding agent to create a mixture; and
(d) means for forming the mixture into a desired shape to create the hybrid cultured meat product.

23. The system of claim 22, wherein the means for decellularizing plant scaffolds comprises one or more containers for holding the decellularizing agents and the plant scaffolds, and a rinsing apparatus for removing the decellularizing agents from the plant scaffolds.
